(19) United States
(12) Patent Application Publication
(10) Pub. No.: US 2024/0000590 A1
(43) Pub. Date: Jan. 4, 2024

(54) IMPLANTABLE DEVICES WITH TRACKING, AND RELATED SYSTEMS AND METHODS

(71) Applicant: Merit Medical Systems, Inc., South Jordan, UT (US)

(72) Inventors: John Hall, Bountiful, UT (US); Wayne Mower, Bountiful, UT (US); Christopher Cindrich, Highland, UT (US)

(21) Appl. No.: 18/343,510

(22) Filed: Jun. 28, 2023

Related U.S. Application Data
(60) Provisional application No. 63/367,459, filed on Jun. 30, 2022.

(51) Int. Cl. A61F 2/90; A61F 2/48

(52) U.S. Cl. CPC A61F 2/90; A61F 2/482; A61F 2250/0096; A61F 2250/0001; A61F 2250/0085; A61F 2210/0076; A61F 2230/0069

(57) ABSTRACT

Implantable devices—such as stents and rings—that comprise markers for tracking their deployment in a body lumen are disclosed. The markers may be used to monitor progress of expansion of the implantable device, potential migration of the implantable device, and diameters of the body lumen. Markers may be deployed in the body lumen separately from another implantable device to assist tracking of migration of the implantable device. Markers may be placed circumferentially around a wall of a body lumen to track growth of an aneurysm or a stricture.

### RELATED CASES

This application claims priority to U.S. Provisional Application No. 63/367,459, filed on Jun. 30, 2022 and titled “IMPLANTABLE DEVICES WITH TRACKING, AND RELATED SYSTEMS AND METHODS,” which is hereby incorporated by reference in its entirety.

### TECHNICAL FIELD

The present disclosure relates generally to the field of implantable devices comprising markers or tags, and to systems and methods for determining locations of markers within a patient's body. More particularly, some embodiments relate to stents with reflector markers.

### BRIEF DESCRIPTION OF THE DRAWINGS

The written disclosure herein describes illustrative embodiments that are non-limiting and non-exhaustive. Reference is made to certain of such illustrative embodiments that are depicted in the figures, in which:

FIG. 1 is a side view of an embodiment of a marker for implantation within a patient's body, with part of the marker in cross section.

FIG. 2 is a side view of an embodiment of an implantable device for implantation within a patient's body, with a marker disposed on a wall of the implantable device.

FIG. 3 is a side view of an embodiment of an implantable device for implantation within a patient's body, with a plurality of markers disposed on a wall of the implantable device.

FIG. 4 is a side view of a portion of an embodiment of an implantable device, with a marker disposed on a wall of the implantable device, wherein struts of the implantable device serve as antennas of the marker.

FIG. 5 is a perspective view of an embodiment of an implantable device disposed within a body lumen, with markers disposed on a wall of the implantable device.

FIG. 6 is a side view of an embodiment of a marker for implantation within a patient's body, with part of the marker in cross section, wherein a first antenna and a second antenna of the marker each comprise anti-migration features.

FIG. 7 is a partial view of a patient's circulatory system, with an embodiment of a plurality of markers placed on an inside surface of a wall of the patient's descending aorta.

FIG. 8 is a partial view of a patient's digestive system, with an embodiment of an implantable device with markers disposed in the patient's esophagus, and a separate marker placed on an inside surface of a wall of the esophagus.

FIG. 9 is a partial view of a patient's digestive system and skeletal system, with an embodiment of an implantable device with markers disposed in the patient's esophagus.

### DETAILED DESCRIPTION

Stents and other implantable devices can be used by medical professionals to treat patients with certain pathological conditions, such as aneurysms in blood vessels or blockages in the gastrointestinal tract. For example, in a patient with an aortic aneurysm, placement of an endovascular stent at the site of the aneurysm may be indicated to support the wall of the blood vessel. As another example, an esophageal stent may be placed in a patient's esophagus to keep the esophagus open at the site of a stricture.

In some patients, an implantable device may migrate away from the site where the device was initially placed by the medical professional. Migration of a stent or other implantable device can lead to serious complications. For example, if an endovascular stent migrates along the aorta, the stent may close off openings to other arteries, such as the renal arteries. As another example, an esophageal stent may migrate into the stomach. Among the benefits of certain embodiments of the present disclosure are devices, systems, and methods for tracking migration of a stent or other implantable device.

For some patients who undergo a stent placement operation, it may be desirable to monitor deployment of the stent, including the rate and the extent to which the stent opens to a fully expanded state. Among the benefits of certain embodiments of the present disclosure are devices, systems, and methods for monitoring the progress of opening of a stent or other implantable device.

In some patients, after a stent or other implantable device is placed, a medical professional may determine that the device is not in an optimal location in the body lumen, or that an extender may be helpful for more completely treating the condition. Among the benefits of certain embodiments of the present disclosure are devices, systems, and methods for evaluating the deployment of a stent or other implantable device and determining whether an extender may be necessary.

For some patients, a developing condition may warrant monitoring a growing stricture or a growing aneurysm, when placement of a stent might not yet be indicated. For example, a patient may have an aortic aneurysm in early-stage development. Before the aneurysm grows to an extent at which point an endovascular grafting procedure is indicated, a medical practitioner may recommend placement of an implantable device or devices to help monitor growth of the aneurysm. The implantable device or devices may assist the practitioner in tracking changes in the diameter of the aorta at the site of the weakened blood vessel wall. Among the benefits of certain embodiments of the present disclosure are devices, systems, and methods for monitoring the size of a body lumen, including monitoring growth of strictures or aneurysms.

Embodiments herein may aid a patient or the patient's medical practitioner in monitoring deployment of a stent or other implantable device. The stent or implantable device may have one or more markers or similar components on the device. Locations of the marker or markers may be tracked to determine whether the implantable device remains in its original placement in a body lumen, or whether it has migrated. The marker or markers may further be tracked to estimate diameters of the body lumen to evaluate possible growth of an aneurysm or a stricture.

The components of the embodiments as generally described and illustrated in the figures herein can be arranged and designed in a wide variety of different configurations. Thus, the following more detailed description of various embodiments, as represented in the figures, is not intended to limit the scope of the present disclosure, but is merely representative of various embodiments. While various aspects of the embodiments are presented in drawings, the drawings are not necessarily drawn to scale unless specifically indicated.

The phrase “coupled to” is broad enough to refer to any suitable coupling or other form of interaction between two or more entities, including mechanical interaction and/or electrical communication. Thus, two components may be coupled to each other even though they are not in direct contact with each other. The phrases “attached to” or “attached directly to” refer to interaction between two or more entities that are in direct contact with each other and/or are separated from each other only by a fastener of any suitable variety (e.g., an adhesive). The phrase “fluid communication” is used in its ordinary sense, and is broad enough to refer to arrangements in which a fluid (e.g., a gas or a liquid) can flow from one element to another element when the elements are in fluid communication with each other.

The terms “proximal” and “distal” are opposite directional terms. For example, the distal end of a device or component is the end of the component that is furthest from the practitioner during ordinary use. The proximal end refers to the opposite end, or the end nearest the practitioner during ordinary use.

References to approximations are made throughout this specification, such as by use of the term “substantially.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers. For example, where the term “substantially perpendicular” is recited with respect to a feature, it is understood that in some embodiments the feature may have a precisely perpendicular configuration.

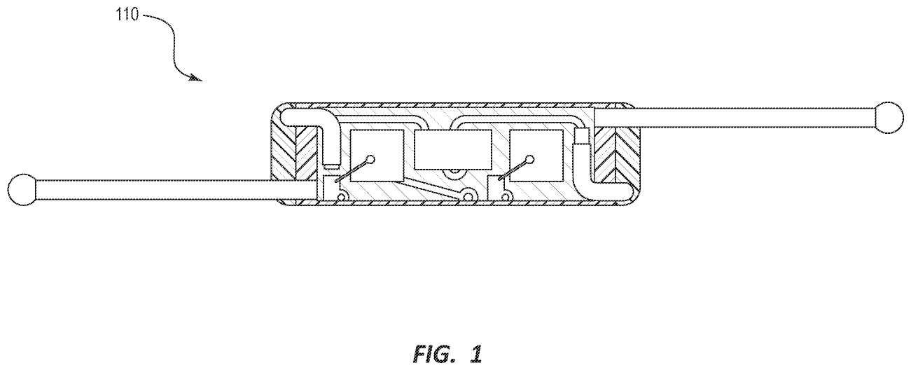

FIG. 1 depicts an embodiment of a marker 110 for implantation within a patient's body. In some embodiments, the marker 110 comprises an energy converter configured to transform energy pulses from an energy source into electrical energy. In some embodiments, the energy converter comprises a photodiode. In some embodiments, the energy pulses include pulses of infrared light. The photodiode may transform the infrared light into electrical current. In some embodiments, the energy pulses may be radiofrequency pulses or some other electromagnetic energy pulses. In some embodiments, the energy pulses may be ultrasonic pulses.

In some embodiments, the marker 110 comprises a switch. The switch may be coupled to the energy converter, so as to be in electrical communication with the energy converter. The switch may have an open state and a closed state. The switch may open or close in response to electrical current from the energy converter. In other words, the electrical energy generated by the energy converter may cause the switch to either open or close. The switch may be a transistor, such as a metal-oxide-semiconductor field-effect transistor. As discussed further below, the switch may be configured to open and close to modulate signals reflected by antennas of the marker 110 to a signal receiver.

In some embodiments, the marker 110 comprises a first antenna and a second antenna. The antennas may comprise elongate members, such as elongate wires or rods. The antennas may comprise an electrically conductive material, such as metal. The antennas may be coupled to the switch, so as to be in electrical communication with the switch. For example, the first antenna may be connected to a first terminal of the switch, and the second antenna may be connected to a second terminal of the switch. Upon closure of the switch, the first terminal and the second terminal may become electrically coupled, thus bringing the first antenna and the second antenna into electrical communication with each other. Upon opening of the switch, the first terminal and the second terminal may become electrically isolated, thus breaking the electrical communication between the first and second antennas. In some embodiments, the switch may be configured differently than described above, for example, such that opening the switch brings the first and second antennas into electrical communication, and closing the switch breaks such electrical communication.

The marker 110 may be configured to respond to a signal. For example, in some embodiments, the marker 110 may reflect a signal emitted from a transmitter such that a signal receiver can detect the reflected signal. As another example, in some embodiments, the marker 110 may transmit an identifying signal in response to receiving an interrogating signal.

For embodiments in which the marker 110 reflects a signal, the antennas of the marker 110 may be configured to reflect energy pulses. In some embodiments, the energy pulses reflected by the antennas of the marker 110 are radar signals. The reflection of energy pulses by the antennas may be different for open configurations of the switch than for closed configurations of the switch. In other words, the antennas may reflect signals in a first fashion when the antennas are electrically isolated from each other, whereas the antennas may reflect signals in a second fashion when the antennas are in electrical communication with each other. The difference between the first fashion of reflection and the second fashion of reflection may be detectable by a receiver of the reflected signals. In this way, the receiver may be able to detect and identify the marker 110. Stated differently, the signature of the marker 110 in a switch-closed state may be different from the signature of the marker 110 in a switch-open state. By modulating the switch, and thereby modulating the signature of the marker 110, a medical practitioner may enable a signal receiver to detect reflections of a signal and determine a location of the marker 110. Various techniques may be used to determine a location of the marker 110.

In some embodiments, such as embodiments wherein the reflected signal is a radar signal, timing information associated with the signal may be used to determine a range of the marker 110 from a signal receiver or signal transmitter. The range together with other spatial information, such as angular positions, may be used to determine a location of the marker 110.

In some embodiments, multiple signal receivers in different locations may be used to receive the reflected signals. Spatial information associated with the marker 110 in relation to each of the multiple receivers may be used to determine a location of the marker 110, such as by using a triangulation process.

The energy pulses sent from the energy source may be coded to have a particular sequence to give the marker 110 a unique identifying pattern. In this way, the energy source can modulate the energy pulses to assign unique patterns to multiple markers 110. In other words, the opening and closing of the switch of a first marker 110 may have a different pattern or sequence than the opening and closing of the switch of a second marker 110. Thus, each marker 110 out of multiple markers 110 may be uniquely detected and their individual locations may be determined.

For embodiments in which the marker 110 transmits an identifying signal in response to receiving an interrogating signal, the marker 110 may include a radio-frequency identification (RFID) tag or a similar device. The tag may be passive or active. The identifying signal may contain timing information as well as unique data to differentiate the marker 110 from other markers 110 among multiple markers 110.

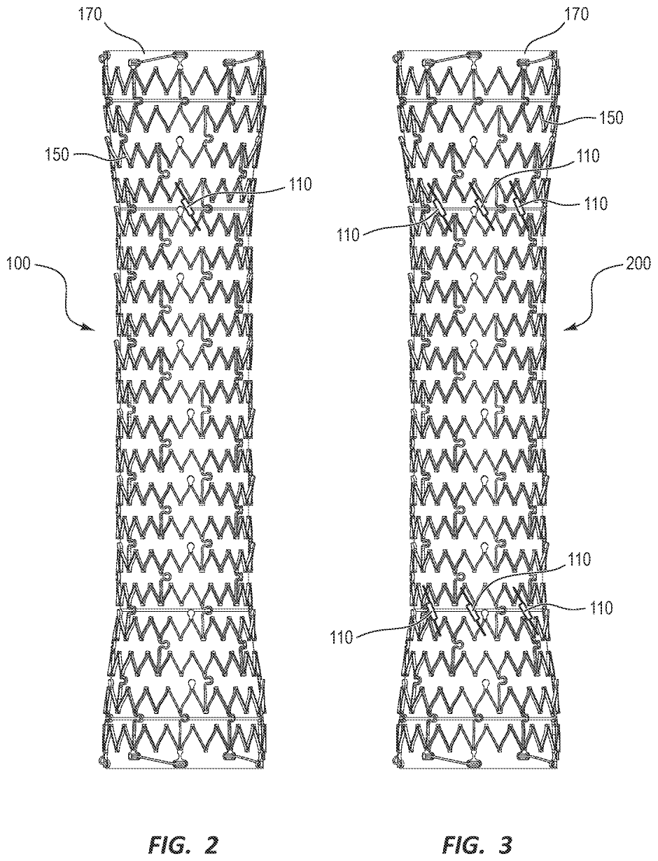

FIG. 2 depicts an embodiment of an implantable device 100 for implantation in a patient's body. The implantable device 100 may be a stent. The implantable device 100 may have a scaffolding of struts 150 configured to collapse to a narrow configuration for insertion into the patient's body along an insertion catheter, and configured to expand to a wide configuration to conform with an internal surface of a wall of a body lumen upon deployment. In some embodiments, the implantable device 100 may have a generally cylindrical shape, such as that shown in FIG. 2. In some embodiments, the implantable device 100 may have a ring shape, similar to the embodiment described in connection with FIG. 5 below.

The implantable device 100 may have a cover 170 applied to the scaffolding of struts 150. In some embodiments, the cover 170 may be disposed on an inside of the scaffolding of struts 150. In some embodiments, the cover 170 may be disposed on an outside of the scaffolding of struts 150. The cover 170 may be formed from a polymeric material configured to collapse and expand with the scaffolding of struts 150.

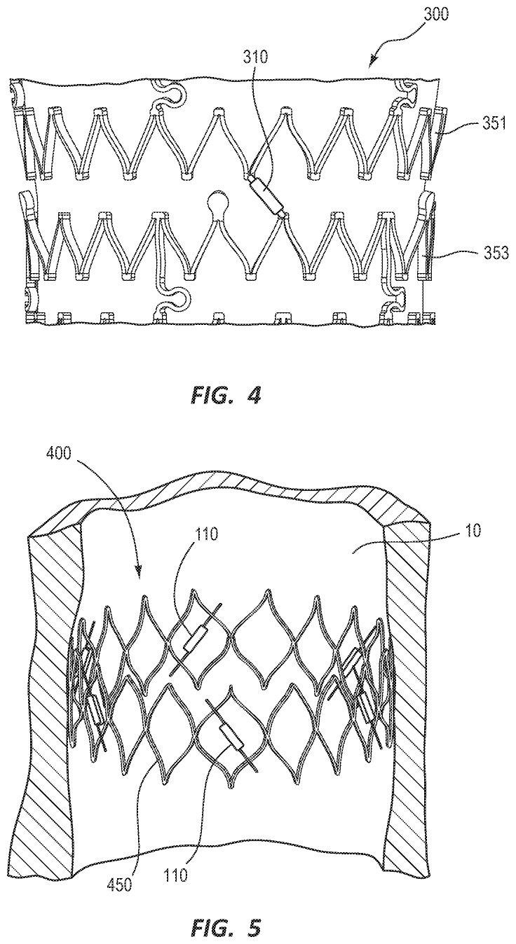

The implantable device 100 may have a marker 110 disposed on a wall of the implantable device 100. In some embodiments, the marker 110 may be disposed on an outside of the scaffolding of struts 150. In some embodiments, the marker 110 may be disposed on an inside of the scaffolding of struts 150. Antennas of the marker 110 may be electrically isolated from the scaffolding of struts 150 of the implantable device 100. As discussed below in connection with FIG. 4, in some embodiments, one or more antennas of the marker 110 may be in electrical communication with the scaffolding of struts 150 of the implantable device 100.

The implantable device 100 may be inserted into a body lumen to assist with a body function. For example, the implantable device 100 may be an esophageal stent to help keep the patient's esophagus open for proper digestion. As another example, the implantable device 100 may be an endovascular stent to support the walls of a blood vessel and prevent or treat an aneurysm. The marker 110 disposed on the implantable device 100 may be used to monitor the deployment of the implantable device 100. For example, by determining the location of the marker 110 within a patient's body, a medical practitioner may be able to determine whether the implantable device 100 has been correctly positioned within the body lumen, and whether the implantable device 100 has migrated after the initial placement.

FIG. 3 depicts an embodiment of an implantable device 200. The implantable device 200 may be a stent. The implantable device 200 resembles the implantable device 100 described above in certain respects. Accordingly, like features are designated with like reference numerals, with the leading digits incremented. For example, the embodiment depicted in FIG. 3 is an implantable device 200 that may, in some respects, resemble the implantable device 100 of FIG. 2. Relevant disclosure set forth above regarding similarly identified features thus might not be repeated hereafter. Moreover, specific features of the implantable device 100 and related components shown in FIG. 2 might not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the implantable device 200 and related components depicted in FIG. 3. Any suitable combination of the features, and variations of the same, described with respect to the implantable device 100 and related components illustrated in FIG. 2 can be employed with the implantable device 200 and related components of FIG. 3, and vice versa. This pattern of disclosure applies equally to further embodiments depicted in subsequent figures and described hereafter, wherein the leading digits may be further incremented.

In the embodiment depicted in FIG. 3, the implantable device 200 has multiple markers 110 disposed on a wall of the implantable device 200. In some embodiments, multiple markers 110 may be placed around the wall of the implantable device 200 in a circumferentially offset pattern. In some embodiments, multiple markers 110 may be placed on the wall of the implantable device 200 in a longitudinally offset pattern. As shown in FIG. 3, there may be a first plurality of markers 110 around the circumference of the implantable device 200 adjacent or near a proximal end of the implantable device 200, and a second plurality of markers 110 around the circumference of the implantable device 200 adjacent or near a distal end of the implantable device 200. In some embodiments, additional markers 110 may be included at other longitudinal and/or circumferential locations on the implantable device.

In some embodiments, each of the markers 110 is disposed on an outside surface of the implantable device 200. In some embodiments, each of the markers 110 is disposed on an inside surface of the implantable device 200. In some embodiments, some of the markers 110 are disposed on an outside surface of the implantable device 200, and some of the markers 110 are disposed on an inside surface of the implantable device 200.

In some embodiments, at least one of the markers 110 on the implantable device 200 comprises an energy converter, a switch, and antennas to reflect signals to a signal receiver to detect locations of the markers 110. In some embodiments, at least one of the markers 110 on the implantable device 200 comprises an RFID tag to transmit signals with identifying data in response to interrogating signals, wherein the signals with identifying data are used to detect locations of the markers 110.

Using an implantable device 200 with a plurality of markers 110 may give a medical practitioner additional benefits beyond those described above in connection with the implantable device 100. For example, having multiple markers 110 around a circumference of the implantable device 200 may allow the practitioner to monitor the progress of expansion of the implantable device 200, and thus estimate an internal diameter of the body lumen. Further, having multiple markers 110 at multiple longitudinal locations along the implantable device 200 may give the practitioner increased ability to monitor the progress of expansion of the implantable device 200, and to track potential migration of the implantable device 200, among other parameters of interest associated with deployment of the implantable device 200.

FIG. 4 depicts a portion of an embodiment of an implantable device 300, with a marker 310 disposed on a wall of the implantable device 300. The implantable device 300 may be a stent. The marker 310 resembles the marker 110 in certain respects. For example, the marker 310 may be configured to respond to a signal. In some embodiments, the marker 310 may reflect a signal emitted from a transmitter such that a signal receiver can detect the reflected signal. The marker 310 may comprise an energy converter, a switch, a first antenna, and a second antenna. In some embodiments, the marker 310 may transmit an identifying signal in response to receiving an interrogating signal. The marker 310 may comprise an RFID tag. The relevant descriptions of the features of the marker 110 apply equally to the features of the marker 310.

In embodiments with a scaffolding of struts that comprises an electrically conductive material, such as metal, a portion of the scaffolding may perform the function of an antenna. In the depicted embodiment, the marker 310 uses struts 351, 353 of the implantable device 300 as antennas. In some embodiments, the struts 351, 353 may each serve as only a portion of an antenna. For example, a strut may be in electrical communication with another member, such as an elongate wire, in which case the strut and the other member together may serve as the antenna. In other embodiments, the struts 351, 353 may serve as the full antennas for the marker.

In some embodiments, the first antenna may comprise a strut or other portion of the scaffolding of struts, while the second antenna may comprise an elongate member, such as a wire or rod. In some embodiments, the first antenna may comprise one portion of the scaffolding, while the second antenna may comprise a different portion of the scaffolding.

FIG. 5 depicts an embodiment of an implantable device 400 disposed within a body lumen 10. In some embodiments, such as the depicted embodiment, the implantable device 400 comprises a ring having an expandable mesh 450. The expandable mesh 450 may have a collapsed configuration for delivery of the implantable device 400, and an expanded configuration with a generally cylindrical shape. In some embodiments, the implantable device 400 comprises a ring having a scaffolding of struts. The scaffolding of struts may have a collapsed configuration for delivery of the implantable device 400, and an expanded configuration with a generally cylindrical shape. In some embodiments, the implantable device 400 comprises a ring having an expandable band. The expandable band may have a collapsed configuration for delivery of the implantable device 400, and an expanded configuration with a generally cylindrical shape.

In the expanded configuration, the ring of the implantable device 400 may conform with an inner surface of the wall of the body lumen 10. In some embodiments, the implantable device 400 may comprise a shape-memory material such as nitinol. The shape-memory material may help the implantable device 400 transition from the collapsed configuration to the expanded configuration.

The implantable device 400 may have a cover applied to the ring. In some embodiments, the cover may be disposed on an inside surface of the ring. In some embodiments, the cover may be disposed on an outside surface of the ring. The cover may be formed from a polymeric material configured to collapse and expand with the ring.

The implantable device 400 may have multiple markers 110 disposed on a wall of the ring. For example, the multiple markers 110 may be coupled to an outside or an inside surface of the expandable mesh 450. In some embodiments, the implantable device 400 may have two markers 110. In some embodiments, the implantable device 400 may have three markers 110. In some embodiments, the implantable device 400 may have four markers 110. In some embodiments, the implantable device 400 may have five markers 110. In some embodiments, the implantable device 400 may have six markers 110. In some embodiments, the implantable device 400 may have seven markers 110. In some embodiments, the implantable device 400 may have eight markers 110. In some embodiments, the implantable device 400 may have more than eight markers 110. Some of the markers 110 may be disposed on an outside surface of the implantable device 400. Some of the markers 110 may be disposed on an inside surface of the implantable device 400.

In some embodiments, the markers 110 of the implantable device 400 are the same as the markers 110 of the implantable devices 100, 200 depicted in FIGS. 2-3. In some embodiments, the markers 110 may be similar to the markers 310 of the implantable device 300 depicted in FIG. 4, wherein at least a portion of one or more antennas of some of the markers 110 include a portion of the struts or mesh of the ring of the implantable device 400. Thus, the description above of the markers 110, 310 in connection with FIGS. 1-4 applies to the markers 110 of the implantable device 400. Like the markers 110, 310 of the implantable devices 100, 200, and 300, the markers 110 of the implantable device 400 may be configured to respond to a signal. In some embodiments, at least one of the markers 110 on the implantable device 400 comprises an energy converter, a switch, and antennas to reflect signals to a signal receiver to detect locations of the markers 110. In some embodiments, at least one of the markers 110 on the implantable device 400 comprises an RFID tag to transmit signals with identifying data in response to interrogating signals, wherein the signals with identifying data are used to detect locations of the markers 110.

A medical practitioner may deploy the implantable device 400 in the body lumen to monitor the size (e.g., the internal diameter) of the passageway through the body lumen 10. For example, the practitioner may seek to monitor growth of a stricture in the body lumen 10, or the practitioner may seek to monitor growth of an aneurysm in the body lumen 10. The practitioner may deploy the implantable device 400 into the body lumen 10, thereby deploying a plurality of markers 110 circumferentially around a wall of the body lumen 10. Using methods described above in connection with FIG. 1 to modulate the plurality of markers 110 and to detect reflected signals or identifying signals from the markers 110, the practitioner may determine locations of each of the markers 110 of the implantable device 400. Based on the determined locations of the markers 110, an internal diameter of the body lumen 10 can be estimated. For example, the practitioner may compare locations of at least two of the plurality of markers 110 relative to each other to estimate relative distances between the markers 110. This location and relative distance information may be sufficient to estimate a diameter of the (at least partially) expanded implantable device 400. Additionally, known properties of the implantable device 400, such as the circumferential positions of each marker 110 on the implantable device 400, may be helpful to the practitioner to estimate the diameter of the (at least partially) expanded implantable device 400. From the estimate of the diameter of the implantable device 400, the practitioner may thereby deduce an internal diameter of the body lumen 10. In the event that a stricture, for example, occludes a portion of the body lumen 10, the location information of the markers 110 may yield dimensions of an irregular cross-sectional shape of the body lumen 10. The location information may help the practitioner to identify a position (such as a circumferential position) of the stricture within the body lumen 10.

In some circumstances, an implantable device 400 with more markers 110 may yield more accurate and/or more complete dimensional information for the body lumen 10 than would be given by an implantable device 400 with fewer markers 110.

The practitioner may compare the estimated internal diameter of the body lumen 10 with a reference diameter to evaluate an extent of opening of the implantable device 400. In other words, the practitioner may seek to determine an extent to which the implantable device 400 has expanded.

The practitioner may repeat at least some of the actions described above after a period of time to monitor changes of the internal diameter of the body lumen 10. In this way, the practitioner may monitor, for example, changes in the size of a stricture or in the size of an aneurysm in the body lumen 10.

The methods described herein in connection with the implantable device 400 depicted in FIG. 5 are also applicable to the implantable device 200 depicted in FIG. 3. Thus, a practitioner may use the implantable device 200 to monitor an internal diameter of a body lumen. However, in some circumstances, the implantable device 400 may be a preferable choice of implantable device over the implantable device 200. For example, in some circumstances, migration of the implantable device 200 may give more cause for concern than migration of the implantable device 400. This may be the case if there is a higher risk of blocking passages, such as blood vessel branches in the patient's vasculature, with the implantable device 200 than with the implantable device 400.

Relatedly, in some circumstances, a medical practitioner might not prescribe placement of an endovascular stent in a patient's descending aorta when an aortic aneurysm is only small. The practitioner may recommend placement of a ring, such as the implantable device 400, to monitor potential growth of the aneurysm. In this way, the aneurysm may be monitored over time without the need for the patient to return to the clinic to undergo additional operations to check up on the progression of the aneurysm. This may save substantial medical risk and financial expense to the patient.

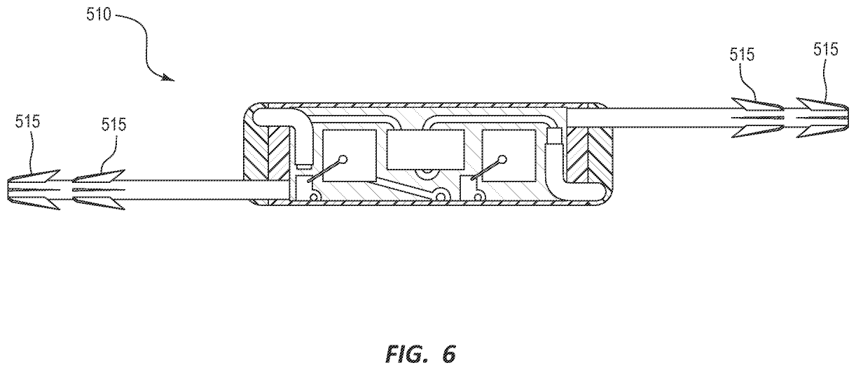

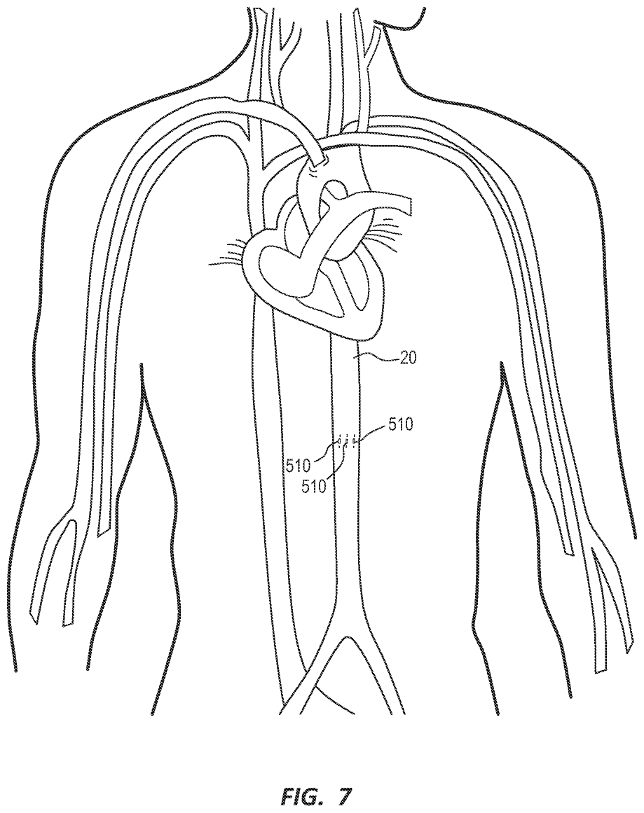

FIG. 6 depicts an embodiment of a marker 510 for implantation within a patient's body. The marker 510 resembles the marker 110 in certain respects. For example, the marker 510 may be configured to respond to a signal. In some embodiments, the marker 510 may reflect a signal emitted from a transmitter such that a signal receiver can detect the reflected signal. The marker 510 may comprise an energy converter, a switch, a first antenna, and a second antenna. In some embodiments, the marker 510 may transmit an identifying signal in response to receiving an interrogating signal. The marker 510 may comprise an RFID tag. The relevant descriptions of the features of the marker 110 apply equally to the features of the marker 510.

As depicted in FIG. 6, the antennas of marker 510 may have anti-migration features 515. The anti-migration features 515 may be barbs, hooks, protrusions, or other features configured to couple with tissue and retain the marker 510 in place at an insertion site in a body lumen.

As described further below, a medical practitioner may elect to deploy one or more markers 510 into a body lumen separate from another implantable device.

FIG. 7 depicts part of a patient's circulatory system, with a plurality of markers 510 placed circumferentially around an internal surface of a wall of the patient's descending aorta 20. The markers 510 may be used to estimate an internal diameter of the descending aorta 20.

Methods employed by a medical practitioner to monitor the diameter of the descending aorta 20 may be similar to the methods described above in connection with the implantable device 400 depicted in FIG. 5. For example, a medical practitioner may obtain a plurality of markers 510, wherein some or all of the markers 510 comprise anti-migration features 515. The practitioner may deploy the plurality of markers 510 circumferentially around a wall of the descending aorta 20 to monitor the size (e.g., the internal diameter) of the passageway through the descending aorta 20. For example, the practitioner may seek to monitor a stricture in the descending aorta 20, or the practitioner may seek to monitor growth of an aneurysm in the descending aorta 20. Using the methods described above in connection with the markers 110 of FIG. 1, the practitioner may modulate the plurality of markers 510, detect reflected signals or identifying signals from the markers 510, and determine locations of each of the markers 510. Based on the determined locations of the markers 510, an internal diameter of the descending aorta 20 can be estimated. For example, the practitioner may compare locations of at least two of the plurality of markers 510 relative to each other to estimate relative distances between the markers 510. The practitioner may be able to use the location and relative distance information to estimate an internal diameter of the descending aorta 20. In the event that an aneurysm is developing in the descending aorta 20, for example, the location information of the markers 510 may yield dimensions of an irregular or enlarged cross-sectional shape of the descending aorta 20.

While FIG. 7 depicts an embodiment within the scope the above-described method carried out in a descending aorta 20, in some embodiments, the method may be performed similarly in other body lumens. Therefore, the relevant description given above of the methods associated with markers 510 applies equally to using markers 510 to estimate diameters of other body lumens.

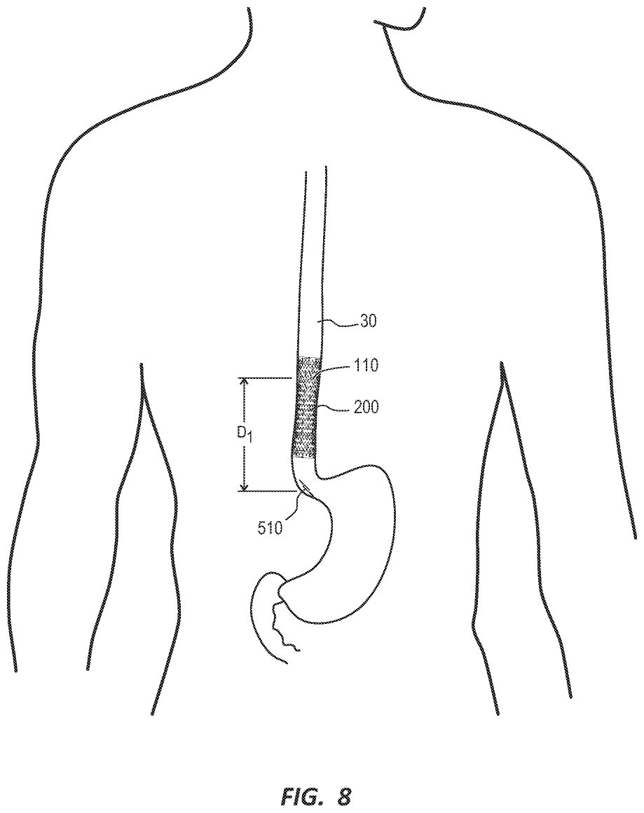

FIG. 8 depicts part of a patient's digestive system, with an implantable device 200 deployed in the patient's esophagus 30, and a marker 510 implanted on an inside surface of the wall of the esophagus 30. The marker 510 is distinct from markers 110 of the implantable device 200. While the implantable device 200 is depicted, some other implantable device, such as one of implantable devices 100, 300, and 400, could similarly be used.

A medical practitioner may employ various methods to monitor deployment of the implantable device 200. For example, if the implantable device is an esophageal stent, the practitioner may track the progress of expansion of the stent over a period of time, such as several days. The practitioner may use methods to determine locations of markers 110 on the implantable device 200 and estimate a diameter of the esophagus 30, using techniques similar to those described above in connection with the implantable device 400 depicted in FIG. 5.

As another example of monitoring deployment of the implantable device 200, the practitioner may track potential migration of the implantable device 200. The practitioner may do this by monitoring a distance between the implantable device 200 and a reference location. The marker 510 implanted on the wall of the esophagus 30 may serve as a landmark, and its location may be the reference location. As depicted in FIG. 8, the practitioner may measure a distance D₁ between a marker location (e.g., the location of one of the markers 110 on the implantable device 200) and the reference location (e.g., the location of the marker 510). The distance D₁ may be measured by detecting the marker location and the reference location, and calculating a difference between these locations.

The detection of the marker location and the reference location may be carried out using some of the methods described above. For example, the practitioner may transmit a first energy (such as infrared light pulses or pulses of another electromagnetic energy) to the implantable device 200. The first energy may be used to open or close a switch on a marker 110 of the implantable device 200 to modulate signals reflected by the marker 110. The practitioner may simultaneously transmit a second energy (such as radar pulses or pulses of another electromagnetic energy) to the implantable device 200. The second energy (which is different from the first energy in at least one characteristic, such as wavelength) may be reflected off of the marker 110 and detected by a signal receiver. The same or a similar technique may be used to detect the reference location by focusing the first and second energies on the marker 510 implanted on the wall of the esophagus 30.

Over a period of time, the practitioner may monitor changes in the value of D₁ to determine whether the implantable device 200 has migrated away from its initial placement location.

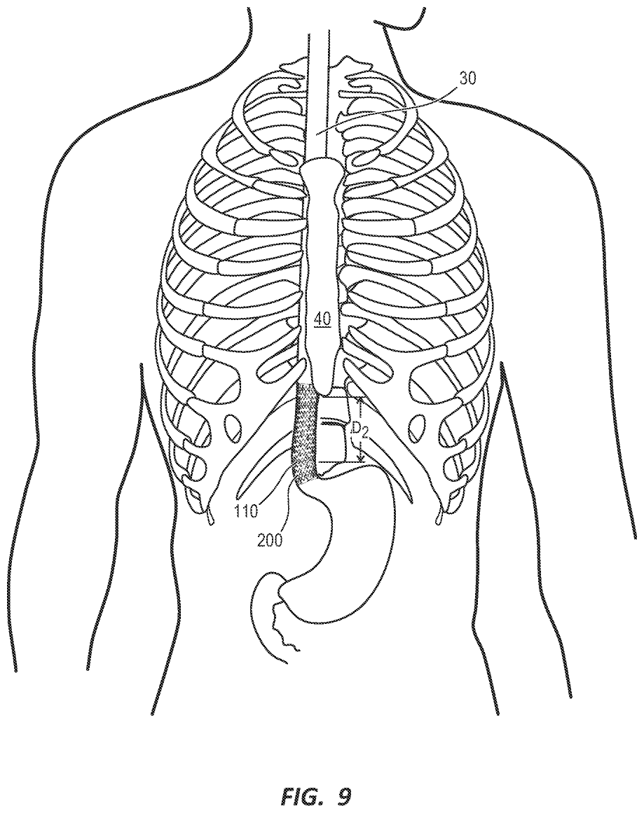

FIG. 9 depicts parts of a patient's digestive system and skeletal system, with an implantable device 200 disposed in the patient's esophagus 30. While the implantable device 200 is depicted, some other implantable device, such as one of implantable devices 100, 300, and 400, could similarly be used.

In contrast with the embodiment depicted in FIG. 8, a medical practitioner may use a landmark other than a marker placed inside the same body lumen as the implantable device 200. For example, in some embodiments, the practitioner may compare the location of the implantable device 200 (determined by detecting the location of a marker 110 disposed on the implantable device 200) with a fixed landmark on the patient's body, such as an anatomical landmark. In the embodiment depicted in FIG. 9, the anatomical landmark is the xiphoid process of the sternum 40. The practitioner may measure a distance D₂ between the xiphoid process of the sternum 40 and a marker 110 on the implantable device 200. Over a period of time, the practitioner may monitor changes in the value of D₂ to determine whether the implantable device 200 has migrated away from its initial placement location.

Other landmarks may be used than the xiphoid process of the sternum 40 depicted in FIG. 9. In some embodiments, the practitioner may use another anatomical landmark, such as another bone, or such as the navel. In some embodiments, the practitioner may implant a separate marker, such as a marker 110, subcutaneously under the epidermis or dermis at a site near the implantable device 200. The location of any of these possible landmarks may serve as the reference location from which the practitioner may track potential migration of the implantable device 200.

In some embodiments, the medical practitioner may determine that an implantable device (such as a stent) previously deployed in a body lumen is insufficient for treating the patient's condition. For example, the implantable device may be misplaced, or it may have insufficient length. This determination may be based on location information acquired through one or more of the methods disclosed herein. The practitioner may determine that an extender is warranted, and may proceed to deploy the extender into the body lumen and to couple the extender to the implantable device. The extender may comprise markers, in similar fashion to the implantable devices disclosed herein.

In some embodiments, a patient may track location information from the markers using a mobile device, such as a tablet or smartphone. The mobile device may generate the necessary signals to interrogate the markers, may receive the responses from the markers, and may perform the required calculations to determine locations of the markers. Remote monitoring in this way may help reduce or even obviate the need for the patient to visit a medical practitioner's clinic to obtain status updates on the deployment of implantable devices and/or markers.

In some embodiments, an implantable device may comprise a flow rate sensor, a pressure sensor, a chemical composition sensor, a pH sensor, and/or the like.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated by one of skill in the art with the benefit of this disclosure that in the above description of embodiments, various features are sometimes grouped together in a single embodiment, figure, or description thereof for the purpose of streamlining the disclosure. This method of disclosure, however, is not to be interpreted as reflecting an intention that any claim requires more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

Recitation in the claims of the term “first” with respect to a feature or element does not necessarily imply the existence of a second or additional such feature or element. It will be apparent to those having skill in the art that changes may be made to the details of the above-described embodiments without departing from the underlying principles of the present disclosure.

We claim:

1. An implantable device configured to be deployed within a body lumen, the device comprising:
a scaffolding of struts; and
one or more markers disposed on a wall of the device, wherein each of the one or more markers is configured to respond to a signal.

2. The implantable device of claim 1, wherein the device has a generally cylindrical shape.

3. The implantable device of claim 1, further comprising a cover applied to the scaffolding of struts, and
wherein the cover comprises a polymeric material.

4. The implantable device of claim 3, wherein the cover is disposed on an outside of the scaffolding of struts.

5. The implantable device of claim 1, wherein at least one of the one or more markers is disposed on an outside surface of the device.

6. The implantable device of claim 1, wherein at least one of the one or more markers comprises:
an energy converter configured to transform energy pulses from an energy source into electrical energy;
a switch with an open state and a closed state, wherein the switch is coupled to the energy converter such that the electrical energy from the energy converter causes the switch to open or close; and
a first antenna and a second antenna, the first and second antennas coupled to the switch,
wherein the switch is configured to open and close to modulate signals reflected by the first and second antennas to a signal receiver.

7. The implantable device of claim 6, wherein the first antenna comprises a first elongate member and the second antenna comprises a second elongate member.

8. The implantable device of claim 6, wherein the first antenna comprises at least a portion of a strut of the scaffolding of struts.

9. The implantable device of claim 6, wherein the energy converter comprises a photodiode.

10. The implantable device of claim 1, wherein at least one of the one or more markers comprises an RFID tag.

11. An implantable device configured to be deployed within a body lumen, the device comprising:
a ring; and
a plurality of markers disposed on a wall of the ring, wherein each of the plurality of markers is configured to respond to a signal.

12. The implantable device of claim 11, wherein the ring comprises an expandable mesh.

13. The implantable device of claim 11, further comprising a cover applied on an outside surface of the ring, and
wherein the cover comprises a polymeric material.

14. The implantable device of claim 12, wherein at least one of the plurality of markers comprises:
an energy converter configured to transform energy pulses from an energy source into electrical energy;
a switch with an open state and a closed state, wherein the switch is coupled to the energy converter such that the electrical energy from the energy converter causes the switch to open or close; and
a first antenna and a second antenna, the first and second antennas coupled to the switch,
wherein the switch is configured to open and close to modulate signals reflected by the first and second antennas to a signal receiver, and
wherein the first antenna comprises at least a portion of the expandable mesh.

15. A method for monitoring a diameter of a body lumen, the method comprising:
obtaining a plurality of markers;
deploying the plurality of markers circumferentially around a wall of a body lumen of a patient; and
comparing locations of at least two of the plurality of markers relative to each other to estimate a diameter of the body lumen.

16. The method of claim 15, wherein at least one of the plurality of markers comprises an anti-migration feature.

17. The method of claim 15, wherein the plurality of markers is disposed on an implantable device configured to expand within the body lumen to conform with an inner surface of the wall of the body lumen.

18. The method of claim 17, further comprising comparing the estimated diameter to a reference diameter to evaluate an extent of opening of the implantable device.

19. The method of claim 15, wherein at least one of the plurality of markers comprises an energy converter, a switch, a first antenna, and a second antenna, and
wherein further comprising modulating the at least one of the plurality of markers, the modulating comprising transmitting a first energy signal to the at least one of the plurality of markers to open or close a switch of the at least one of the plurality of markers.

20. The method of claim 15, further comprising determining the locations of the at least two of the plurality of markers, the determining comprising transmitting a second energy signal to the at least two of the plurality of markers and detecting reflections of the second energy signal from the at least two of the plurality of markers.
